(19) United States
(12) Patent Application Publication
(10) Pub. No.: US 2006/0240449 A1
(43) Pub. Date: Oct. 26, 2006

(54) Methods and compositions for preparation of biological samples

(76) Inventors: Ronald C. McGlennen, Edina, MN (US); David J. Olson, Minnetrista, MN (US); Elaine M. Stores, New Brighton, MN (US); Aaron M. Franks, Eden Prairie, MN (US); Naomi M. Williamson, Fridley, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/336,665

(22) Filed: Jan. 19, 2006

Related U.S. Application Data
(60) Provisional application No. 60/645,442, filed on Jan. 19, 2005.

(51) Int. Cl. C12Q 1/68; C12N 1/08

(52) U.S. Cl. 435/6; 435/270

(57) ABSTRACT

Methods and compositions for preparation of biological samples are disclosed. The methods include a prelysis step and a lysis step to make the cellular DNA available for further processing, amplification or analysis. The prelysis step includes the addition of a prelysis reagent to the cells. The prelysis reagent may include an enzyme to facilitate the disruption of the cells. The lysis step includes the addition of a lysis reagent to at least a portion of the prelysis reagent and cells.

### CROSS-REFERENCE TO RELATED APPLICATIONS

The present application claims priority from a United States Provisional Patent Application having Ser. No. 60/645,442 filed Jan. 19, 2005 the disclosure of which is hereby incorporated by reference in its entirety.

### BACKGROUND OF THE INVENTION

1. Field of Invention

The present inventions relate to the preparation of biological samples and, more particularly, to methods and compositions to provide access to the genetic material of biologic samples for analysis, amplification and/or further manipulation.

2. Description of the Related Art

Almost all eukaryotic cells have a nucleus that contains the DNA of the cell. In addition, prokaryotic organisms also have DNA or RNA as the template for replication of its complement of genes. Molecular diagnostics utilizes the nucleic acid, derived from a variety of biologic samples, to detect and characterize gene structure and gene expression. The initial steps in the performing many molecular diagnostic tests involves the extraction and isolation of nucleic acid, including deoxyribonucleic acid (DNA) as well as ribonucleic acid (RNA). Extraction of DNA and RNA typically involves disruption of the cell, thereby exposing the nucleic acid that lies within.

The extraction of DNA and/or RNA has historically been a multi-step process. The initial step in the process of extraction of DNA typically involves the disruption of the cell membrane or in the case of, bacteria or plant cells, the cell wall. A variety of methods are used to lyse cells, some involving mechanical disruption. These can include sonication and pulverization with microbeads comprised of carbon, silica and other inert materials, and other mechanical and physical methods.

The subsequent steps in nucleic acid extraction and isolation typically involve the differential removal of aqueous phase soluble proteins and associated lipid. Frequently, this is achieved by the addition of high concentrations of a neutral salt such as potassium chloride or sodium acetate. High concentrations of these salts denature most protein and cause them to be less soluble. The addition of high salt, often complemented by cold temperature incubation, can cause protein and lipid to precipitate from solution. Centrifugation of the resulting mixture separates the precipitate and the remaining aqueous solution, comprised of salt, small protein and nucleic acid, both DNA and RNA. Several methods and commercial products for DNA and RNA purification are based on these described principles of cell lysis with detergent and differential removal of protein and lipid through a “salting out” process. Differential isolation of DNA or RNA isolation is typically based on the use of salt solutions with more neutral (for DNA) or acidic (for RNA) pH.

The final step in nucleic acid purification typically involves the concentration of solubilized DNA or RNA through precipitation by the addition of ethyl or isopropyl alcohol. The addition of these alcohols to a solution of nucleic acid in a high salt solvent will cause DNA or RNA to precipitate from solution. Nucleic acid is retrieved from this precipitation step by high speed centrifugation and removal of the liquid phase. The resulting DNA or RNA pellet can then be readied for use in a chemical assay by rehydration, usually with the addition of a small volume of a neutral buffer such as Tris-EDTA or water. In protocols that start with biologic materials that are freshly procured and that are transported and stored in ambient temperatures, the resulting DNA or RNA is of a purity such that the characterization of that purity by means of ultraviolet light absorption approximates values of free nucleotides dissolved in a water based solution. Nucleic acid prepared in this manner is typically of sufficient quality to be used in conjunction with other bioanalytic methods such as Southern transfer and the polymerase chain reaction. Moreover, nucleic acid samples proving not to be of sufficient purity for these latter techniques can again be re-extracted by simply repeating the above listed steps, followed by alcohol precipitation and re-hydration

Many biological samples are fixed for purposes of examination, analysis and other purposes. The process of cell or tissue fixation, generally involves the permanent denaturation of cellular proteins, and the cross-linking of proteins within the context of cellular structures so as to preserve the approximate shape and distribution of these structures as they exist when the cells are viable. Frequently, tissues samples are fixed through a process of incubation in a solution of formalin (fomaldehyde 1-5%) in a neutral pH salt buffer, for a time dependent of the size and volume of the sample. Biologic samples collected as single or aggregated collections of cells are fixed in various ways, but most commonly in mixtures of formalin and methyl or ethyl alcohol. Fixation of cells for cytologic analysis can involve the fixation of cells in one of the alcohol solutions followed by application of those cell suspensions to an inert membrane which in turn is then applied to a glass microscope slide. This basic method of sample collection and re-deposition onto glass slides has resulted in a significant improvement in the quality and reliability of such common diagnostic procedures, such as, for example, a Pap smear. Unfortunately, attempts at extraction and isolation of nucleic acids from samples that are fixed prior to these procedures typically leads to a nucleic acid preparation of significantly lower purity. Accordingly, a need exists for methods and compositions which produce a high purity nucleic acid preparation.

Several protocols describe methods for extraction of DNA and RNA from formalin fixed tissues and cytology preparations. Generally, these methods are similar to the methods described above involving salt precipitation of protein and alcohol isolation of nucleic acid. In each case, the methods involve the serial treatment of sample with various reagents, followed by heat incubation and centrifugation. In practice, the volumetric transfer of sample from a primary tube to a second tube, and the associated action of centrifugation and precipitation leads to a proportional loss in yield of DNA. Accordingly, each serial transfer is estimated to reduce DNA yield by 10%. In addition, a significant amount of labor is associated with such protocols involving multiple sample transfers and various mechanical steps. Thus, a need exists for a simple and rapid method for extracting DNA from cells and tissues fixed with formalin, alcohol or other chemical fixing reagents, and for cells and tissues embedded in paraffin or other similar materials.

### SUMMARY OF THE INVENTION

The present inventions provide for efficient extraction of DNA from various sources of biological samples. The compositions and methods are particularly suited for extracting DNA from chemically fixed cells or tissues. In other aspects, compositions and methods in accordance with the present inventions are particularly suited for extracting DNA from fresh or chemically fixed cells and tissues which have been embedded in paraffin or other materials. The resulting DNA can have a purity sufficient for purposes of genetic analyses and molecular diagnostic testing. The compositions and methods of the present inventions may be particularly adapted to improve the ease of processing of biological samples subjected to fixation prior to use in molecular genetic testing.

In one aspect, the present inventions may provide compositions and methods for nucleic acid isolation from a variety of biologic samples based on the use of a single lysis reagent. In other aspects, the present inventions may provide compositions and methods for nucleic acid isolation from a variety of biologic samples based on the use of a prelysis reagent and a lysis reagent. In an aspect of the present inventions, the prelysis reagent and the lysis reagent are the same reagent. The present inventions may provide novel methods using commercially available lysis reagents and other lysis reagents adapted to various sample types for the extraction and preparation of DNA for subsequent molecular genetic analyses. The utility of these disclosed compositions and methods in various combinations can improve on prior methods used for the isolation of DNA from biological samples not only in the performance of certain assays, but also in the ease of use and ability to scale this procedure to process large volumes of samples and adapt to automated systems. The present inventions may also be integrated into a more holistic system for molecular diagnostic testing. The holistic system may include processing specific materials that guide the use of this protocol in a series of molecular genetic assays and integration into an internet-based system that organizes workflow, analytic processes and involves online assay interpretation. The compositions and methods may simplify aspects of molecular genetic testing making the testing more easily usable by smaller and less experienced laboratories.

The compositions and methods of the present inventions may be formulated and configured to extract DNA from fresh or fixed cell and tissue samples in a two step process. The methods in accordance with the present inventions may provide an improved lysis and extraction techniques, which employ one or more reagents that disrupt and/or solubilize cellular membranes or cell walls, denature and fragment cellular protein and solubilize cellular, viral and bacterial DNA.

In one aspect, the present inventions may include a lysis reagent for disrupting the cell membrane. In another aspect, the present inventions may also include a prelysis reagent. The prelysis reagent and the lysis reagent facilitate the disruption of the cell membrane. This may be achieved by the addition of detergents or use of hypo-osmotic solvents such as water, methanol or weak salt solutions as the prelysis reagent and/or the lysis reagent. The prelysis reagent may further contain various enzymes or other components to more easily facilitate the disruption of the cells. The enzymes may include a protease, such as Proteinase K, for example, or a lysozyme. Typically, the lysis reagent and, if used, the prelysis reagent for lysis of nucleated cells are an aqueous solution including Tris-EDTA and sodium dodecyl-sulfate (SDS) at quantities ranging from 0.5-10% weight/volume. SDS serves as the detergent, which solubilizes the lipid bilayer, effectively creating disruption of the membrane. The lysis reagent may be a commercially available lysis solution, such as the lysis reagent marketed under the tradename microLYSIS by Microzone Ltd., having a location in Haywards Heath, West Sussex, UK or the lysis reagent marketed under the tradename Lyse-N-Go Reagent by Pierce Biotechnology having a location in Rockford, Ill., USA. The compositions and methods in accordance with the present inventions may provide comparable or improved yield of DNA which may be subsequently useable in a PCR reaction or related gene chemistry applications.

In one aspect, the present inventions may use microLYSIS or Lyse-N-Go Reagent as the lysis reagent and, if present, the prelysis reagent to lyse cellular membranes or cell walls and achieve improved solubilization of the component protein and lipid constituents of cellular structures. The combined effect of multiple neutral and nonionic detergents, along with heat activated reactants in microLYSIS causes cell membranes, whether fixed or unfixed, to dissolve. Treatment of fixed cells with varying amounts of microLYSIS reagent results in nearly complete dissolution of cells following incubations as short as 1 to 2 minutes. Typically, the final concentration of the microLYSIS reagent for such treatments is 1× as defined by the manufacturer.

In one aspect, the method for cellular lysis procedure may include two or more of the following steps: Cellular samples collected fresh are sedimented by gravity or centrifugation followed by the removal of the associated collection media. In the case of fresh sample collection, the media, which may be a nutrient media, or a balanced buffered salt solution such as phosphate buffered saline or lactate ringer, can be decanted or removed by pipetting. For samples fixed in formaldehyde based fixative or for commercial Pap collection medias containing alcohols, the media is removed, and the cells washed once or twice with a wash solution such as Tris-EDTA, Tris-EGTA, phosphate buffered saline, HEPES, HEPES-EDTA, HEPES-EGTA, or water. The wash solution may have a substantially neutral pH. The wash solution is typically removed by decanting after the cells have been pelleted. A volume of prelysis reagent of between 1 and 5 times the volume of the resulting cell pellet is typically added to the sample and the cells are resuspended. In one exemplary aspect, a volume of concentrated microLYSIS reagent may be added such that the resultant suspension dilutes the microLYSIS to a concentration of 1×. A proteinase or lysozyme may then be added to the cells suspended in the prelysis reagent. This step is referred to as prelysis. Prelysis is a requirement of these protocols in that the subsequent dilution of the cell sample is made easier and more uniform. This is particularly true for samples that are fixed and those treated with the separate step of passage through a polysaccharide gradient, which causes the samples to clump and become sticky. In the case of the latter process, the prelysis step is preceded by a wash in either water or a buffered solution to remove the adherent carbohydrate from the cell surface.

The resulting mixture is vortexed to generate a “cell slurry”. A volume as small as 2 μL is added to another volume of microLYSIS. This resulting low-density cell suspension is then incubated at varying temperature steps designed to activate components, complete cell lysis and protein degradation. Following this, the resulting cellular homogenate containing the DNA can be assayed directly.

With some prelysis reagents and lysis reagents, the extraction may be sensitive to the volume of reagent relative to the volume of the cell sample. In one embodiment of the invention, the estimated size or volume of the cell pellet comprising the sample is compared to a template guide that lists the corresponding correct volume of diluent or lysis reagent solutions required for optimal extraction. This guide permits the visual comparison of the pellet size with various standards shown on the template to permit an adequate approximation of the volume of reagent to be used to resuspend the cell pellet. In another embodiment, the addition of a volume of prelysis or lysis reagent equal or larger than the volume of the cell pellet is adequate for the prelysis step of the present invention. The addition of this volume of lysis reagent is termed the prelysis, because in a subsequent transfer of a small amount of this mixture is then diluted and resuspended in a larger volume of lysis reagent to achieve a 3:1 lysis reagent to cell volume, which is the second and final step in the lysis procedure.

In another aspect of the invention, the prelysis and lysis steps may be applied to fragment of tissue, including such typical samples as sections of solid organ tissue (lymph node, liver skin) or bone cores containing bone marrow. In this case, the prelysis step may include any of a variety of mechanical disruption processes using a vortexer, a tissue homogenizer or an ultrasonic probe for example. The prelysis step may further include the addition of a micro-bead suspension to assist in breaking up the tissue into smaller fragments during the process of mechanical disruption. This step may increase the available surface area of the sample for subsequent lysis and fragmentation of the tissue mass. This is followed by dilution with lysis reagent and the addition of up to 1 mg/ml of a neutral protease such as proteinase K or a lysozyme for example. The addition of the latter augments this digestion of the protein which is further denatured and solubilized during incubation.

In another aspect of the invention, the prelysis and lysis steps may be applied to tissues fixed with formalin or comparable fixatives and then subsequently embedded in paraffin. Such preparation are typical of tissue sample procured for morphologic examination in pathology laboratories. In this case, the prelysis step is accompanied with the addition of heat to melt the paraffin, followed by dilution and the addition of up to 1 mg/ml of a neutral protease such as proteinase K. The addition of the latter augments this digestion of the protein which is denatured further, and better solubilized during the lysis incubation.

### DETAILED DESCRIPTION OF THE INVENTION

The present invention provides methods and compositions for extraction of DNA from various sources of biologic samples. Methods and compositions in accordance with the present inventions may be utilized for extraction of high molecular weight genomic DNA, viral genomic DNA and bacterial genomic and plasmid DNA. The DNA may be extracted from fresh, fixed or paraffin embedded cells or tissue. The invention is especially adapted to improve the ease of use of samples subjected to fixation prior to use in molecular genetic testing. The inventions may permit extraction of DNA in two steps: prelysis step and a lysis step. In one aspect, a single lysis reagent may be used for both the prelysis step and the lysis step. In another aspect, a prelysis reagent is used for the prelysis step and a lysis reagent is used for the lysis step. The use of both steps may permit the extracted DNA to be suitable for use in a broad variety of molecular genetic assays and several common genetic assay methods. The methods described herein are generally simpler than earlier described methods in that there is reduction of wash steps prior to cell lysis; and there is no need for the process of isolating DNA by centrifugation and precipitation. The inventions provide methods which may be implemented on various integrated instrument platforms and within Web-Enabled DNA testing systems, such as, for example, that disclosed in U.S. Pat. App. Pub. No. 2004/0014097 A1 by Ronald McGlennen et al. the disclosure of which is hereby incorporated by reference in its entirety.

Before lysing the cells of a cell or tissue sample, the cells may be concentrated. Cell samples are sedimented or pelleted by gravity or centrifugation followed by the removal of the associated media. The media may be a nutrient media or a balanced buffered salt solution such as phosphate buffered saline or lactate ringer for example. After sedimentation, the media can typically be easily decanted or removed by pipetting. For fixed cell samples the media may be the fixative in which the cells are fixed. Typically, the cells are fixed in formaldehyde based fixative or an alcohol based fixative such as some commercial Pap collection media for example, the media is first removed.

The fixed cells are then washed once or twice with a wash solution such as Tris, EDTA or EGTA, phosphate buffered saline, HEPES or HEPES-EDTA or EGTA buffer, or water. After washing, the cells are typically pelleted by, for example, centrifugation and the wash solution is pipetted or decanted away. This step is also important for cells treated with the separate step of passage through a polysaccharide gradient, which causes the samples to clump and become sticky. In the case of the latter process, lysis is preceded by a wash in either water or a buffered solution to remove the adherent carbohydrate from the cell surface.

The methods in accordance with the present inventions typically include a prelysis step and a lysis step. The prelysis step in the context of the present inventions involves the addition of a prescribed volume of a lysis reagent or a prelysis reagent to a pellet of tissue or cells which may permit the formation of a more heterogeneous slurry of cells that are non-adherent. The prelysis reagent and lysis reagent are typically selected to be compatible with the subsequent use of the DNA made available by the prelysis and lysis steps. In one aspect, the prelysis reagent and lysis reagent may be selected to be compatible with the polymerase chain reaction (PCR) methodologies. The lysis reagent and/or prelysis reagent may contain other buffering compounds in an amount sufficient to maintain the pH of the composition in the range of 6.0 to about 9.0.

The prelysis reagent and/or the lysis reagent facilitate the disruption of the cell membrane and cell lysis. This may be achieved by the addition of non-ionic and/or ionic detergents. The detergents used in the lysis reagent and/or prelysis reagent may include various ionic and non-ionic detergents alone and in combination and in Tris-EDTA or other buffer solutions well known in the art. Particularly, crude cell lysates are suitable for PCR when obtained by protein digestion and membrane lysis with detergents such as SDS, Nonidet P-40 (NP-40), Tween 20, or Laureth 12 alone or the combination of Tween 20 and NP40 among other combinations. In addition or alternatively, the prelysis reagent and lysis reagent may use of hypo-osmotic solvents such as water, methanol or weak salt solutions as the prelysis reagent and/or the lysis reagent to at least assist in lysing the cells. The lysis reagent and/or prelysis reagent may also contain other components, such as cell membrane altering compounds, such as vancomycin or polymyxin B, RNAases and defoamer. The prelysis reagent may further contain various enzymes or other components to more easily facilitate the disruption of the cells. The enzymes may include a protease, such as Proteinase K, for example, or a lysozyme. Typically, the lysis reagent and, if used, the prelysis reagent for lysis of nucleated cells are an aqueous solution including Tris-EDTA and sodium dodecyl-sulfate (SDS) at quantities ranging from 0.5-10% weight/volume. SDS serves as the detergent, which solubilizes the lipid bilayer, effectively creating disruption of the membrane. The lysis reagent may be a commercially available lysis solution, such as the lysis reagent marketed under the tradename microLYSIS by Microzone Ltd., having a location in Haywards Heath, West Sussex, UK or the lysis reagent marketed under the tradename Lyse-N-Go Reagent by Pierce Biotechnology having a location in Rockford, Ill., USA.

The prelysis step includes adding a volume of prelysis reagent to a sample of washed cells. Typically, the volume of prelysis reagent is between 1 to 5 times that of the sample of washed cells. In one aspect, the washed cells are resuspended to a concentration of between 1 million and 1.5 million cells per milliliter. In one embodiment of the invention, the estimated size or volume of the cell pellet comprising the sample is visually compared to a template guide that lists the corresponding correct volume of diluent or lysis reagent solutions required for optimal extraction. In another embodiment, the addition of a volume of lysis reagent equal or larger than the volume of the cell sample serves as prelysis step of methods in accordance with the present inventions. Cell lysis may only partial during the prelysis step of the present methods, and is the means by which the sample is mixed and made more fluid for the subsequent pipetting step into a larger and diluting volume of the lysis reagent. The addition of this volume of lysis reagent is termed the prelysis, because in a subsequent transfer of a small amount of this mixture is then diluted and resuspended in a larger volume of lysis reagent to achieve a 3:1 lysis reagent to cell volume, which is the second and final step in the lysis procedure. Multiples of this volume proportion may also be used in the lysis procedure.

After addition of the prelysis reagent, the mixture is vortexed for 10-20 seconds, and then heated at 95° C. for 1-20 minutes. The heating may function to denature any proteases added for cellular disrupter or that may be otherwise present in the sample and would be detrimental to later use of the lysed cells such as for example for a PCR reaction. The result of this step is a near homogeneous mixture of cells and lysis reagent. This sample is now ready for transfer to the second step, dilution in lysis reagent.

Transfer of the prelysis mixture from the primary vessel to the diluted lysis reagent reaction vessel is a crucial step in this procedure and central to this invention. The transfer of cells previously fixed, e.g., liquid Pap collected samples, containing large epithelial cells, is made difficult due to the sticky, adherent nature of these cells and the mechanical limitations related to aspirating fixed cells through small apertures. The prelysis step makes these very heterogenous samples more uniform, and permits a more accurate transfer of sample to the diluted lysis reagent step.

In certain aspects of the present inventions, 1-2 μL of the prelysis sample are transferred to the diluted lysis reagent vessel. In other aspects, up to 20 μL or more of the prelysis sample may be transferred the diluted lysis reagent vessel. A common lysis reagent vessel may include a plastic 96 well microtiter plate, capable strips of microcentrifuge/PCR tubes, or comparable multiplexed vessels. The invention also includes the volumetric transfer from the primary vessel to an enclosed system with multiple reaction wells connected via microfluidic conduits.

Transfer of the prelysis mixture is an important step in the present methods. In one embodiment, the volumetric transfer is achieved using a single channel or a multi-channel manual pipetting instrument. In another embodiment, the pipetting is achieved using a robotic system, such as illustrated in FIG. 5 using the Eppendorf epMotion 5070 robot, or similar robotic device.

The lysis step is based on the appropriate dilution of the prelysis cell mixture with lysis reagent. The preferred embodiment of this dilution and the subsequent volume for the lysis reaction is 1 part cells to 19 parts lysing solution. Integer multiples of this ration of sample to lysing solution are considered. Under these conditions, and involving the necessary incubation of the diluted sample at 65-95° C. for 10-20 minutes results in complete cellular lysis and homogenization of the sample mixture without the need for mechnical mixing, pelleting or precipitation. The resulting homogeneous mixture contains high molecular weight chromosomal, viral circle, bacterial chromosomal or plasmid DNA of sufficient purity so as to be suitable for PCR or comparable gene chemistry reactions and assays.

The present invention allows for the addition of a quantity of a nonspecific proteinase, such as 1 μL of a stock of 10 mg/ml Proteinase K to the diluted lysed sample to enhance protein fragmentation. This latter adaptation is useful for samples proving to have fibrous and acellular protein carry over.

In another embodiment of the invention, the prelysis and lysis steps may be applied to fragment of tissue, including such typical samples as sections of solid organ tissue (lymph node, liver skin) or bone cores containing bone marrow. In these cases, the prelysis is accompanied by more vigorous vortexing agitation, or with mechanical disruption with a tissue homogenizer, ultrasonic probe source or with the addition of a microbead suspension. The purpose of this step is to increase the available surface area of the sample for subsequent lysis and fragmentation of the protein mass. This is followed by dilution with lysis reagent and the addition of up to 1 mg/ml of a neutral protease such as proteinase K. The addition of the latter augments this digestion of the protein which is further denatured and solubilized during incubation.

In another embodiment of the invention, the prelysis and lysis steps may be applied to tissues fixed with formalin or comparable fixatives and then subsequently embedded in paraffin. Such preparations are typical of tissue sample procured for morphologic examination in pathology laboratories. Cells and biological samples embedded in paraffin may undergo a deparaffinization process as will be recognized by those skilled in the art. In one aspect, the prelysis step is accompanied with the addition of heat to melt the paraffin as a component of the deparaffinization process. After removal of the paraffin, the cells may be resuspended in a prelysis reagent. A neutral protease such as proteinase K may be included or added to the prelysis reagent at a concentration of to 1 mg/ml. The addition of the latter augments this digestion of the protein which is denatured further, and better solubilized during the lysis incubation.

The current invention embodies the use of the lysis reagent in PCR based molecular genetic assays. The quality of the lysis reagent is such that there is nominal inhibition of the PCR process. Hence the DNA and the lysed mixture can be added as a preferred source of DNA template to a host of conventional and novel PCR based assays. In a preferred embodiment of this invention, the lysed cell mixture is added to the PCR assay for HPV detection. In another embodiment, the lysed cell mixture is added to the PCR reaction for a multiplexed Chlamydia and Neisseria gonorrheae assay. In another embodiment, the lysed cell mixture is added directly to a assay for mutation detection in the cystic fibrosis or factor V or prothrombin or methylene tetrahydrofolate reductase genes. In yet another assay, the lysed cell mixture is added directly to a PCR based assay to detect group B streptococcus or herpes simplex virus. It is envisioned that the current invention may use DNA derived from this lysis procedure for a host of diagnostic and experimental molecular genetic assays.

The present invention may be assembled as a kit for extracting DNA from fixed and fresh cells. The kit would contain lysis reagents and the template for determining the volume of the cell pellet. Other kit components may include but are not limited to the following: buffers for washing fixed cells and embedded cells and tissues, and tubes and microwell plates.

These methods can correspondingly be adapted to various automated and miniaturized instrument platforms. In one aspect of the invention, the lysis protocol is adapted to a robotic pipetting device. In this case, the addition of liquid for the wash steps, the addition of a lysis reagent for the prelysis step, the subsequent transfer of the cell mixture to a second vessel and subsequent dilution of that mixture with a lysis reagent in the final steps is achieved by a device that is a uses a programmable pipettor to deliver and collect sample automatically. One example of this is the adaptation of the Eppendorf epMotion 5070 robotic station to this procedure. The invention encompasses the specific programming and instrument modifications for other commercial robotic platforms to accomplish the same.

One aspect of the invention is the adaptation of the lysing procedure to a miniaturized microfluidic platform that can perform the processes of mixing, dilution and incubation of the cellular sample and lysis reagent on a spinning disc containing microfluidic channels, ports and values. In this case, cell sample is added to an open or valved port on the disc. The operation of spinning the disc delivers the cell sample to a different location on the disc where a pre-measured quantity of wash or lysis solution is mixed to the fluid cell sample. Continued spinning or local actuation of the disc results in the mixing and incubation in the prelysis steps. Subsequent delivery of the prelysis mixture to the same or different position on the disc results in the lysis step. Similarly, adaptation of this lysis protocol to other miniaturized platforms, including ones based on MEMS or microelectromechnical systems technology, such as those disclosed by Furcht et. al., are considered part of this invention.

The present inventions may facilitate the use and commercialization of various integrated genetic testing platforms. Such a platform would include the capability to add the biologic sample, with or without the necessary washing directly onto a device where the subsequent steps of prelysis, lysis and various analytic procedures would take place. Such systems have been envisioned in U.S. Pat. Nos. 6,303,288 and 6,054,277 by Furcht et. al., the disclosures of which are hereby incorporated by reference, where method of nucleic acid extraction, the polymerase chain reaction and detection and measurement of the subsequent DNA amplification product occur on a single silicon chip. Other apparatus and methodologies for process integration have been developed including those by Caliper Technologies, Nanogen Corporation, Roche Diagnostics, Applied Biosystems, GeneOhm Sciences and 3M.

The current invention also encompasses aspects of process integration involving the adaptation of the protocols herein across differing instruments and platforms. In one aspect of the invention, the lysis procedure for fixed and unfixed samples is integrated by means of a internet based web-enabled system that provides the necessary organization and workflow tools to enhance the efficiency and simplicity of this set of procedures. Such as web-enabled DNA testing system has been disclosed, and includes a subsystem for sample accessioning, the production of a sample worklists, batch load and the subsequent organization of the DNA samples onto PCR worksheets, placement within reaction plates and the cataloging of samples for the subsequent analysis by gel electrophoresis, direct detection on a rotating disc, capillary electrophoresis or other comparable chemical and physical methods for DNA product analysis.

In another aspect of the present inventions, the compositions and methods may be adapted to prepare the DNA in a sample of cells for subsequent molecular genetics assays. In its present embodiment, the lysis procedure can serve as the source of nucleic acid in the polymerase chain reaction based detection of human papillomavirus, Chlamydia and Neisseria, detection of mutations in the gene for cystic fibrosis and the analysis of common genetic markers of inherited thrombophilia. In these cases, extraction of DNA from a single sample of cells, such as that from alcohol base liquid Pap collection systems such as the commercial preparations sold under the tradenames ThinPrep™ by the Cytyc Corporation having offices in Marlborough, Mass. USA and SurePath® by TriPath Imaging, Inc. having offices in Raleigh Durham, N.C. result in a single source of DNA that can be assayed for one or multiple of the above listed or other.

Correspondingly, the lysis preparation of DNA from samples such as the liquid Pap may be archived for long periods of time without significant degradation. The archived DNA can be used at later times to test for addition molecular genetic markers. One embodiment of the archive storage of the lytic sample is to store the residual volume of nucleic acid sample on the extraction disc, or MEMs chip. Use of the disc or chip can then be assayed for additional molecular genetic markers.

### EXAMPLE 1

In a first exemplary embodiment, up to 5 mL of a sample including fixed cells from a Pap smear in the alcohol based ThinPrep™ fixative is transferred to a 10 mL conical centrifuge tube. Distilled water is added to bring the total volume in each tube up to 10 mL. The fixed cells are then pelletted by centrifugation. The pellet of fixed cells is then resuspended in a volume of water typically between about 35 μL to 3 mL to generate a suspension with between about 1 million and 1.5 million cells per ml. To determine the volume of water to be added, the cell pellet volume is compared to the cell pellet evaluation guide to determine the required dilution volume. The supernatant is decanted and excess water blotted away with a paper towel. The dilution volume of water determined from the cell pellet evaluation guide is added. The prelysis reagent is 100 mM Tris (hydroxymethyl) aminomethane, 500 mM Potassium chloride at pH 8.9-9.0 and may contain 1% Triton X as a stabilizer. 28.75 μL of prelysis reagent is prepared in a prelysis microtiter plate with the addition of 1.25 μL Proteinase K (20 μg/mL). 30 μL of the resuspended cells is then added to the prelysis reagent and Proteinase K. The prelysis microtiter plate is then incubated in a thermal cycler at for 30 minutes at 65 degrees Celsius followed by five minutes at 22 degrees Celsius. To each well of a lysis microtiter plate is added 15 μL of a 1.33× microLYSIS as the lysis reagent and 5 μL of each sample from the prelysis microtiter plate. At this stage, the lysis microtiter plate may be stored at −20 degrees Celsius for later us or used immediately for DNA amplification using a methodology such as polymerase chain reaction.

### EXAMPLE 2

In a second exemplary embodiment, a sample of fixed cells from a Pap smear in an alcohol based SurePath® fixative is centrifuged to form a cell pellet. The cell pellet is transferred to a 10 mL conical centrifuge tube. Distilled water is added as a wash solution to bring the total volume in each tube up to 10 mL and the cells are again pelleted. The cell pellet is then resuspended in a volume of water typically between about 35 μL to 3 mL to generate a suspension with between about 1 million and 1.5 million cells per ml. To determine the volume of water to be added, the volume of the pellet of fixed cells is typically compared to the cell pellet evaluation guide to determine the required dilution volume, which can range from. The supernatant is decanted and excess water blotted away with a paper towel. The dilution volume of water determined from the cell pellet evaluation guide is added. The prelysis reagent is 100 mM Tris (hydroxymethyl) aminomethane, 500 mM Potassium chloride at pH 8.9-9.0 and may contain 1% Triton X as a stabilizer. 28.75 μL of prelysis reagent is prepared in a prelysis microtiter plate with the addition of 1.25 μL Proteinase K (20 μg/mL). 30 μL of the resuspended cells is then added to the prelysis reagent and Proteinase K. The prelysis microtiter plate is then incubated in a thermal cycler at for 30 minutes at 65 degrees Celsius followed by five minutes at 22 degrees Celsius. To each well of a lysis microtiter plate 15 μL of a 1.33× microLYSIS is added as the lysis reagent and 5 μL of each sample from the prelysis microtiter plate. At this stage, the lysis microtiter plate may be stored at −20 degrees Celsius for later us or used immediately for DNA amplification using a methodology such as polymerase chain reaction.

What is claimed is:

1. A method for extracting and isolating DNA from comprising the steps of:
providing fixed cells;
washing the fixed cells in a wash solution to substantially remove a fixative;
removing the cells from the wash solution;
suspending the fixed cells in a prelysis reagent;
disrupting a cellular membrane of the fixed cells;
adding a lysis reagent to the prelysis reagent and the fixed cells; and
transferring an aliquot of the lysis reagent, prelysis reagent and fixed cells into a reaction vessel.
